(19) United States
(12) Patent Application Publication
(10) Pub. No.: US 2025/0073015 A1
(43) Pub. Date: Mar. 6, 2025

(54) A FLOW DIVERTING APPARATUS FOR CHRONIC INFLAMMATION AND LYMPHEDEMA

(71) Applicant: AORTO MEDICAL LLC., TANGIER (MA)

(72) Inventor: Youssef EL AZOUZI, TANGIER (MA)

(21) Appl. No.: 18/728,201

(22) PCT Filed: Jan. 13, 2023
(86) PCT No.: PCT/IB2023/050324

§ 371 Date: Jul. 11, 2024

Related U.S. Application Data
(60) Provisional application No. 63/299,005, filed on Jan. 13, 2022.
(60) Provisional application No. 63/357,119, filed on Jun. 30, 2022.
(60) Provisional application No. 63/388,789, filed on Jul. 13, 2022.

(51) Int. Cl. A61F 2/01

(52) U.S. Cl. CPC A61F 2/014; A61F 2002/016; A61F 2210/0009; A61F 2210/0057; A61F 2210/0071; A61F 2210/0076; A61F 2230/0067; A61F 2230/0069; A61F 2250/0015; A61F 2250/0024; A61F 2250/0037; A61F 2250/0039; A61F 2250/004; A61F 2250/0041; A61F 2250/0046; A61F 2250/0047; A61F 2250/0048; A61F 2250/0054; A61F 2250/0056

(57) ABSTRACT

A flow diverting apparatus includes a stent frame body, and a stent frame extension.

The stent frame body includes an inlet opening, an outlet opening, and a cavity extending from the inlet opening to the outlet opening. The stent frame body is configured to pass a flow of fluid from the inlet opening to the outlet opening through the cavity. The stent frame body includes a first portion having the inlet opening, and a second portion having the outlet opening. The second portion is tapered from one end of the second portion to a first location of the second portion, thereby forming an indented portion having a plurality of pores. The stent frame extension is configured to be disposed between at least a portion of the indented portion and a vessel wall, thereby preventing a migration of vessel wall cells onto the indented portion.

### PRIORITY CLAIM

The present application claims priority to and the benefit of U.S. Provisional Patent Application No. 63/299,005, filed on, Jan. 13, 2022; U.S. Provisional Patent Application No. 63/357,119, filed on, Jun. 30, 2022; and U.S. Provisional Patent Application No. 63/388,789, filed on, Jul. 13, 2022, the entirety of each of which is incorporated herein by reference.

### BACKGROUND

The concept of the flow modulation stent in the treatment of various disease states involves leveraging access of the systemic vascular system throughout the human body as a potential site of therapeutic intervention. Through different readaptation methods, the flow modulation stent can be reconfigured in order to serve as a tool for the prevention or impediment of pathophysiological processes within the context of blood transport. More broadly, diversion of blood components, whether in a selective or non-selective manner can prove vital in the restoration of physiological homeostasis and thus survival as well as quality of life, while avoiding complications and resistance linked to corticosteroid use or more radical methods of therapeutic intervention such as invasive surgery. Stent technology is most traditionally known for its role in reversing conditions of stenosis caused by atherosclerosis and thrombosis. However, opportunities exist for percutaneously treating a myriad of other diseases with one or more flow modulation stents, and thus methods are needed for doing so.

### SUMMARY

The present disclosure provides new and innovative flow diverting apparatus and method for using the same for treating chronic inflammation and lymphedema. In some examples, a flow diverting apparatus is provided. The flow diverting apparatus includes a stent frame body, and a stent frame extension. The stent frame body includes an inlet opening, an outlet opening, and a cavity extending from the inlet opening to the outlet opening. The stent frame body is configured to pass a flow of fluid from the inlet opening to the outlet opening through the cavity. The stent frame body includes a first portion having the inlet opening, and a second portion having the outlet opening. The second portion is tapered from one end of the second portion to a first location of the second portion, thereby forming an indented portion having a plurality of pores. The first location of the second portion is disposed between the one end and the outlet opening or on the outlet opening. The pores of the indented portion are configured to filter white blood cells based on the white blood cells' size and/or shape while allowing red blood cells to pass therethrough. A cross-sectional area of the inlet opening is greater than a cross-sectional area of the outlet opening. The stent frame extension protrudes from at least a portion of a side surface of the stent frame body and protrudes away from the inlet opening. The stent frame extension is configured to be disposed between at least a portion of the indented portion and a vessel wall, thereby preventing a migration of vessel wall cells onto the indented portion.

In some examples, a method of treating and/or preventing chronic inflammation in a patient is provided. The method includes implanting a flow diverting apparatus in a primary arterial vessel of a patient, the flow diverting apparatus including an inlet opening and an outlet opening, wherein the flow diverting apparatus is implanted such that the inlet opening is positioned within the primary arterial vessel upstream an orifice of a secondary branch vessel that branches off of the primary arterial vessel and the outlet opening is positioned at, upstream, or downstream the orifice, wherein the implanted flow diverting apparatus is positioned and configured such that a proportion of inflammation-causing components in blood of the patient in the primary arterial vessel upstream the flow diverting apparatus being directed away from the secondary branch vessel is greater than a proportion of the inflammation-causing components in the blood of the patient in the primary arterial vessel upstream the flow diverting apparatus being directed towards the secondary branch vessel, and wherein the secondary branch vessel leads to a portion of a body of the patient affected with chronic inflammation.

Additional features and advantages of the disclosed apparatus and methods are described in, and will be apparent from, the following Detailed Description and the Figures.

### BRIEF DESCRIPTION OF THE FIGURES

FIG. 1 is a diagram of a flow diverting apparatus according to an example of the present disclosure.

FIG. 2 is a diagram of the flow diverting apparatus of FIG. 1 installed in a primary arterial vessel according to an example of the present disclosure.

FIG. 3 is a diagram of a flow diverting apparatus according to an example of the present disclosure.

FIG. 4 is a diagram of a flow diverting apparatus according to an example of the present disclosure.

FIG. 5A is a diagram of a flow diverting apparatus according to an example of the present disclosure.

FIG. 5B is a diagram of the flow diverting apparatus of FIG. 5A with a stein frame extension of the flow diverting apparatus shown transparent for illustrative purposes.

FIG. 5C is an example cross-sectional view of the flow diverting apparatus of FIG. 5A taken along line C-C according to an example of the present disclosure.

FIGS. 5D-5G are diagrams of pores of a flow diverting apparatus in a different shape and arrangement according to an example of the present disclosure.

FIG. 6A is a diagram of the flow diverting apparatus of FIG. 5A installed in a primary arterial vessel according to an example of the present disclosure.

FIG. 6B is an expanded and cross-sectional view of the area B of FIG. 6A.

FIG. 7A is a diagram of a flow diverting apparatus according to an example of the present disclosure.

FIG. 7B is a diagram of a flow diverting apparatus according to an example of the present disclosure.

FIG. 7C is a cross-sectional view of the flow diverting apparatus of FIG. 7B taken along line D-D according to an example of the present disclosure.

FIG. 8A is another example cross-sectional view of the flow diverting apparatus of FIG. 5A taken along line C-C according to another example of the present disclosure.

FIG. 8B is another example cross-sectional view of the flow diverting apparatus of FIG. 5A taken along line C-C according to another example of the present disclosure.

FIG. 8C is another example cross-sectional view of the flow diverting apparatus of FIG. 5A taken along line C-C according to another example of the present disclosure.

### DETAILED DESCRIPTION OF EXAMPLE EMBODIMENTS

The present application relates to a flow diverting apparatus and method of using the flow diverting apparatus for treating chronic inflammation in different areas of the body. In various examples, the flow diverting apparatus may be a stent, stent-graft, balloon, or other suitable diverter. In some examples, the flow diverting apparatus may be the stent-graft described in International Patent Application Publication No. WO2020/168216, titled FLOW RESTRICTING STENT-GRAFT, filed Feb. 14, 2020, which is herein incorporated by reference in its entirety.

Concerning chronic inflammation, a flow diverting apparatus can be used to selectively filter blood components and ultimately compartmentalize luminal areas within bodily vessels such that one fractional area of the lumen is significantly less concentrated with all or some types of white blood cells relative to the remaining luminal area. Aspects of the present disclosure may provide a flow diverting apparatus that is capable of preventing hematogenous passage of immune cells into the arterial vascular tree of inflamed tissue. For example, a flow diverting apparatus according to the present disclosure may bar leukocytes that participate in persistent and pathological inflammation from accessing and entering the orifice(s) of secondary branch vessels that feed off of a primary vessel, where the flow diverting apparatus is deployed and located.

In some examples, a flow diverting apparatus according to the present disclosure may be constructed to provide a selective barrier that may reroute or divert immune cells as blood passes through the flow diverting apparatus. In some examples, the material from which the flow diverting apparatus is constructed may help provide the selective barrier. For example, the flow diverting apparatus may be constructed of a mesh material. The mesh material may enable certain blood components to pass through while preventing other blood components from passing through. In various aspects, the mesh material may be made of or include a suitable metal, such as cobalt, nitinol (nickel titanium), stainless steel, etc. In some examples, the mesh material may additionally or alternatively be made of or include a fabric material, such as polyester, polytetrafluoroethylene (PTFE), expanded polytetrafluoroethylene (ePTFE), polyethylene terephthalate (PET) or any other suitable biocompatible-grade material or a combination thereof.

In various examples, the material from which the flow diverting apparatus is constructed may have a permeability. For example, a flow diverting apparatus of the present disclosure may include pores. The pores may be small enough in diameter to allow vital blood components, such as red blood cells, platelets, and plasma to pass through, but to prevent significant amounts of larger-sized white blood cells (e.g., white blood cells having a diameter greater than or equal to 8, 9, 10, 11, 12, 13, 14, 15, 16, 17, 18, 19, 20, or 21 μm), such as neutrophils, monocytes, macrophages, granulocytes, and plasma cells, from passing the pores. In some examples, the pore diameter of the flow diverting apparatus material may be in the range of about 8 μm to about 70 μm, for example, about 10 μm to about 50 μm, about 20 μm to about 40 μm, about 25 μm to about 35 μm, or about 8 μm to about 15 μm.

One advantage of using PTFE or ePTFE fabric from a biocompatibility standpoint may involve the ability to homogenize pore sizes across the fabric's entire surface area in addition to customizing globular, circular, or oval pore shapes rather than slits or channels with potentially sharp angles, though other suitable fabrics or metals may be used as stated above. In some examples, the pores in the flow diverter stent-graft fabric may be broadly circular, elliptical, rectangular or square-shaped (with round corners). In other examples, the pores in the flow diverter stent-graft fabric may have any other suitable shapes.

In some examples, a selective barrier fabric that consists of ellipses may be used in a flow diverting apparatus according to the present disclosure for chronic inflammatory conditions that are mainly driven by the migration and infiltration of t or b lymphocytes, seen in cases such as osteoarthritis or subacute and chronic organ transplant rejection. Filtration of lymphocytes away from artery branches supplying affected organs may primarily require selective filtration based on shape rather than size. This is because lymphocytes and red blood cells have approximately the same diameter size of 7-8 μm. Because lymphocytes are globular-shaped and red blood cells are disk-shaped, red blood cells may more easily penetrate an elliptical pore passages through the selective barrier fabric. Thus, risk of ischemic complications to downstream tissues may be minimized while at the same time, major inflammatory drivers, such as lymphocytes may be prevented from accessing artery branches supplying affected tissue.

In some examples, the elliptical pores of the flow diverting apparatus may span the thickness of the selective fabric barrier and may be about 4 to about 36 μm in length and about 8 to about 64 μm in width, for example, to achieve therapeutic purposes. For example, the elliptical pores of the flow diverting apparatus may be about 4 to about 9 μm in length and about 8 to about 16 μm in width, about 9 to about 18 μm in length and about 16 to about 32 μm in width, about 18 to about 27 μm in length and about 32 to about 48 μm in width, or about 27 to about 36 μm in length, and about 48 to about 64 μm in width.

In some examples, various pore shapes may be organized in rows and/or columns along the desired/predetermined section of the stent frame body/stent-graft fabric with the longest side of the ellipses or rectangular pores being oriented perpendicular or parallel to the direction of blood flow (e.g., from the inlet opening to the outlet opening). The pores may be homogenous in shape throughout a flow diverter stent-graft or may have different pore shapes made within the same flow diverter stent-graft.

In some examples, the pore density within a particular flow diverter stent-graft may vary. The relative porosity of the fabric within the porous section of the stent frame body/graft material may be equal to or greater than 40% of total blood flow, for example, equal to or greater than 45%, equal to or greater than 50%, equal to or greater than 55%, or equal to or greater than 60%. In some examples, the pores may be set up to be aligned along an axis with a square, ellipses, circle, or rectangle located directly above or below another. In other examples, the pores may alternatively be set up to be non-aligned along an axis with a square, ellipses, circle, or rectangle located diagonally above or below another.

In some examples, the shape of the flow diverting apparatus may help provide the selective barrier. For example, the flow diverting apparatus may have a tapered structure such that the cross-sectional area of an upstream (inlet) opening of the flow diverting apparatus through which blood enters is larger than the cross-sectional area of a downstream (outlet) portion of the flow diverting apparatus. The tapering, or narrowing, of the flow diverting apparatus may compel a significant amount of blood flow to travel through the stent frame body of the flow diverting apparatus because the tapering/narrowing decreases the cross-sectional area of the flow diverting apparatus' interior through which blood can flow, thereby enabling only a fraction of the blood to flow from the upstream opening through to the downstream opening. Moreover, since the flow diverting apparatus material may help prevent passage of larger blood components, as described above, those larger blood components may be mainly directed through the interior of the flow diverting apparatus and out the downstream opening.

In some examples, an orientation of the flow diverting apparatus when implanted in a patient can help direct the larger blood components leaving the downstream opening, such as to a secondary branch vessel that feeds off of a primary vessel in which the flow diverting apparatus is implanted, based on where the downstream opening is located within the primary vessel. In various aspects, the tapered section of the flow diverting apparatus may take the form of a vortex, cone, hourglass, lobular or a crescent shape. In some examples, the flow diverting apparatus may include a tapering angle (from an axis extending through a length of the flow diverting apparatus) in the range of about 10 degrees to about 90 degrees, for example, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, or about 80 degrees to about 90 degrees.

In some examples, implanting the flow diverting apparatus at particular locations within a patient can help divert blood, or targeted blood components, to certain areas of the body. By targeting larger-sized immune cells, such as neutrophils and monocytes, that play an important role in the innate immunity of the body, and diverting them away from a site of inflammation, the concentration of chemical mediators, such as cytokines, may decrease within the site of inflammation. This may, in turn, subside important pathophysiological hallmarks of inflammation, such as vascular permeability of capillary beds within inflamed tissues. Reduced secretion of chemical mediators from neutrophils may lead to reduced concentration of chemoattractants, which may otherwise cause a large influx of leukocytic migration towards the site of inflammation. In addition, modulation of up-stream innate immunity may also lead to mirror modulation of down-stream adaptive immunity. Thus, local immunosuppressive targeting of innate immunity cells through endovascular means may prove additionally appealing as downstream adaptive immunity is generally recognized as an important factor in the exacerbation of chronic inflammation.

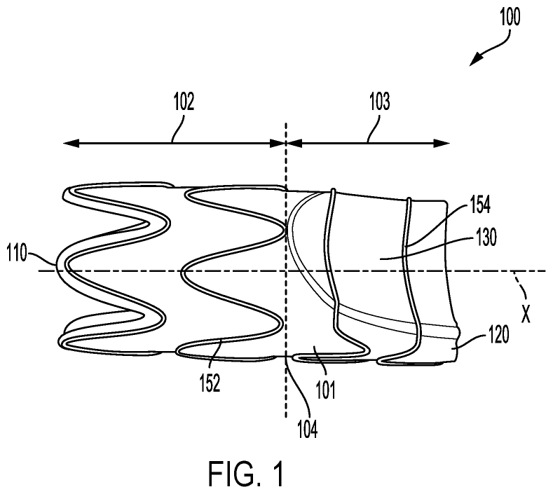

FIG. 1 illustrates a flow diverting apparatus 100 according an example of the present disclosure. The flow diverting apparatus 100 may include a stent frame body 101. The stent frame body 101 may include an inlet opening 110, an outlet opening 120, and a cavity extending from the inlet opening 110 to the outlet opening 120. The stent frame body 101 may be configured to pass a flow of fluid from the inlet opening 110 to the outlet opening 120 through the cavity.

In some examples, the inlet opening 110 (e.g., cross-sectional area thereof) may have a circular shape or any other suitable shape. In some examples, the outlet opening 120 (e.g., cross-sectional area thereof) may be in a shape of a circle, partial circle, oval, or crescent. In other examples, the outlet opening 120 may have any other suitable shape.

The stent frame body 101 may include a first portion 102 having the inlet opening 110 and a second portion 103 having the outlet opening 120. In some examples, the first portion 102 may have a cylindrical shape.

In some examples, the stent frame body 101 may include a tapered/indented portion. For example, the second portion 103 of the stent frame body 101 may be tapered from one (cross-sectional) end 104 of the second portion 103 to a first (cross-sectional) location of the second portion 103, thereby forming an indented portion 130. In some examples, the first location of the second portion 103 may be a location on the outlet opening 120, as shown in FIG. 1. In other examples, the first location may be disposed at any (cross-sectional) location between the one end 104 and the outlet opening 120. In some examples, the second portion 103 may be defined as a portion of the stent frame body 101 that extends from a (cross-sectional) point where the tapering starts to a point where the outlet opening 120 is located, and the first portion 103 is the rest portion of the stent frame body 101 that is not the second portion 102 (e.g., from the inlet opening 110 to the tapering start point).

In some examples, the indented portion 130 may include pores. In some examples, only the indented portion 130 of the stent frame body 101 includes pores, and the other portion of the stent frame body 101 does not include pores. In other examples, the stent frame body 101 may include the pores in any other suitable portion thereof.

The pores may be provided to filter white blood cells based on the white blood cells' size and/or shape while allowing red blood cells to pass therethrough. In this case, most of the white blood cells from the inlet opening 110 may pass through the outlet opening 120. In some examples, a diameter or a cross-sectional area of the inlet opening 110 may be greater than a diameter or a cross-sectional area of the outlet opening 120.

In some examples, the indented portion 130 may be expandable. For example, the stent frame body 101 may be (substantially) in a cylindrical shape when the indented portion 130 is fully expanded.

In some examples, the indented portion 130 may include a curved portion, a concave/convex portion. In some examples, the tapered portion of the flow diverting apparatus 100 may take the form of a vortex, cone, hourglass, lobular or a crescent shape.

In some examples, the tapered/indented portion may include a cross-sectional portion having a shape of crescent, circle, partial circle (e.g., half-moon). In other examples, the tapered/indented portion may include a cross-sectional portion having any other suitable shape (e.g., polygonal shape, regular/irregular shape).

The stent frame body 101 may define a longitudinal axis X extending from the inlet opening 110 to the outlet opening 120. In some examples, an inclined angle formed between the longitudinal axis X and the indented portion 130 may be in a range of about 10 degrees to about 90 degrees, for example, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, or about 80 degrees to about 90 degrees. In some examples, the inclined angle may vary across the indented portion 130. For example, the inclined angle of the indented portion 130 near the end 104 of the second portion 103 may be smaller or greater than the inclined angle near the outlet opening 120. In other examples, the inclined angle of the indented portion may be (substantially) the same across the indented portion 130.

In some examples, the stent frame body 101 may be made with or include polyester, PTFE, ePTFE, PET, or any combinations thereof. In other examples, the stent frame body 101 may be made with or include any other suitable (biocompatible) material (e.g., any other suitable metal material, such as cobalt, nitinol, or stainless steel).

In some examples, the flow diverting apparatus 100 may further include one or more stent frame wires extending around a perimeter of the stent frame body 101. For example, the flow diverting apparatus 100 may include one or more seal frame wires 152, extending around the perimeter of the stent frame body 101. The one or more seal frame wires 152 may extend around the stent frame 101 at its largest perimeter, for instance, outside of the tapered/indented portion (e.g., in the first portion 102).

In some examples, the flow diverting apparatus 100 may further include a plurality of flow-restricting frame wires 154 extending around the perimeter of the stent frame body 101 within the tapered/indented portion. The flow-restricting frame wires 154 may be used to form and maintain the shape of the tapered/indented portion. In some examples, the flow diverting apparatus 100 may include one or more fixation frame wires.

In some examples, the frame wires may have a zig-zag shape, linear shape, curved shape, a concave shape, a convex shape, or a combination of these shapes. In other examples, the frame wires may have any other suitable shape.

In some examples, the frame wires may have a diameter between 0.3 to 0.6 mm. In some examples, the frame wires may be made of or include a shape-memory material, such as nitinol. The shape memory-material may enable the flow diverting apparatus to expand and return to its resting shape during the blood pressure changes of a patient's heart cycling through systolic and diastolic phases. In other examples, the frame wires may be made of or include any other suitable (biocompatible) material. Additional descriptions of the frame wires and the stent frame body are provided in International Patent Application Publication No. WO2020/168216, titled FLOW RESTRICTING STENT-GRAFT, filed Feb. 14, 2020, which is herein incorporated by reference in its entirety.

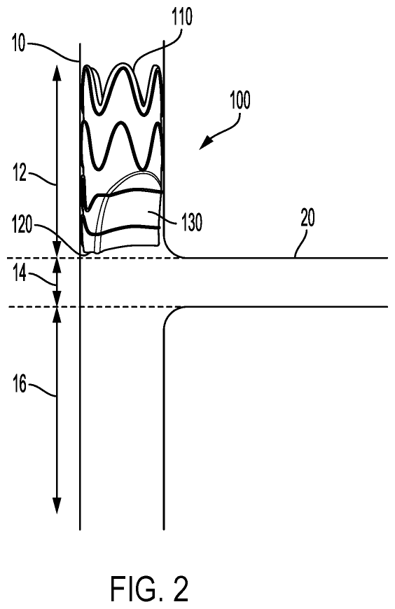

As shown in FIG. 2, the flow diverting apparatus 100 may be placed within a primary arterial vessel 10 such that the inlet opening 110 of the flow diverting apparatus 100 and/or the first portion 102 may be proximal to (upstream) the orifice of an affected secondary branch vessel 20. As used herein, an affected secondary branch vessel may refer to a vessel that branches off a primary arterial vessel and leads to a region of the body affected with chronic inflammation.

The primary arterial vessel 10 may include an upstream portion 12 disposed upstream the orifice of the affected secondary branch vessel 20, a downstream portion 16 disposed downstream the orifice of the affected secondary branch vessel 20, and an intersecting portion 14 disposed between the upstream portion 12 and the downstream portion 16. The intersecting portion 14 is a portion where the primary arterial vessel 10 meets the (orifice of) affected secondary branch vessel 20. In some examples, when the flow diverting apparatus 100 is placed in the primary arterial vessel 10, the inlet opening 110 and/or the first portion 102 of the flow diverting apparatus 100 may be disposed in the upstream portion 12.

In some examples, when the flow diverting apparatus 100 is placed in the primary arterial vessel 10, the indented portion 130 of the stent frame body 101 may be positioned in the upstream portion 12 as shown in FIG. 2. In other examples, when the flow diverting apparatus 100 is placed in the primary arterial vessel 10, the indented portion 130 (at least a portion thereof) of the stent frame body 101 may be disposed in the intersecting portion 14 or the downstream portion 16 of the primary arterial vessel 10.

In some examples, the pores that may form part of the flow diverting apparatus 100 illustrated in FIG. 1 and FIG. 2 may be concentrated on the section of the stent frame body 101 that protrudes along the stent surface/frame into the luminal area of the vessel (e.g., not on the section of the stent frame body 101 that is apposed against the wall of the primary arterial vessel 10).

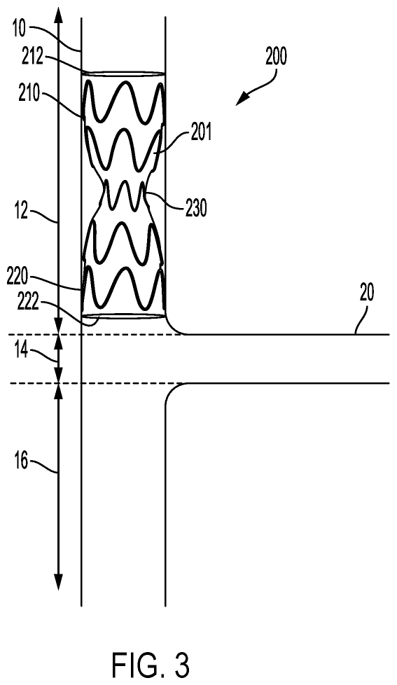

FIG. 3 illustrates another example of a flow diverting apparatus 200 according to an example of the present disclosure. As shown in FIG. 3, the flow diverting apparatus 200 may include a stent frame body 201. The stent frame body 201 may include an inlet section 210 having an inlet opening 212, an outlet section 220 having an outlet opening 222, and a tapered/indented middle section 230 disposed between the inlet section 210 and the outlet section 220. In some examples, the inlet section 210 and the outlet section 220 may have a cylindrical shape. The flow diverting apparatus 200 may have an hourglass shape.

In some examples, the stent frame body 201 may include a cavity extending from the inlet opening 212 to the outlet opening 222. The stent frame body 201 may be configured to pass a flow of fluid from the inlet opening 212 to the outlet opening 222 through the cavity. In some examples, the inlet opening/outlet opening 212, 222 may have a circular or oval shape. In other examples, the inlet opening/outlet opening 212, 222 may have any other suitable shape.

In some examples, when the flow diverting apparatus 200 is placed in the primary arterial vessel 10, the inlet section 210 of the flow diverting apparatus 200 may be located proximal to (upstream) the orifice of the affected secondary branch vessel 20 (e.g., upstream portion 12 of the primary arterial vessel 10).

In some examples, when the flow diverting apparatus 200 is placed in the primary arterial vessel 10, the middle indented section 230 of the flow diverting apparatus 200 may be disposed in the upstream portion 12 of the primary arterial vessel 10, as shown in FIG. 3. In other examples, when the flow diverting apparatus 200 is placed in the primary arterial vessel 10, the middle indented section 230 of the flow diverting apparatus 200 may be disposed in the intersecting portion 14 or the downstream portion 16 of the primary arterial vessel 10.

In some examples, when the flow diverting apparatus 200 is placed in the primary arterial vessel 10, the outlet section 220 of the flow diverting apparatus 200 may be disposed in the upstream portion 12 of the primary arterial vessel 10, as shown in FIG. 3. In other examples, when the flow diverting apparatus 200 is placed in the primary arterial vessel 10, the outlet section 220 of the flow diverting apparatus 200 (or at least a portion of the outlet section 220) may be disposed in the intersecting portion 14 or the downstream portion 16 of the primary arterial vessel 10.

In some examples, the indented section 230 may include a curved portion, a concave/convex portion. In some examples, the indented section 230 of the flow diverting apparatus 200 may take the form of a vortex, cone, hourglass, lobular, or a crescent shape. In some examples, the indented section 230 may include a cross-sectional portion having a shape of crescent, circle, or partial circle (e.g., half-moon). In other examples, the tapered/indented portion may include a cross-sectional portion having any other suitable shape (e.g., polygonal shape, regular/irregular shape).

In some examples, when the indented section 230 (e.g., a cross section thereof) has a crescent shape, the implanted flow diverting apparatus 200 may be oriented such that the crescent shaped tapered/indented section 230 is fixated against the opposing vessel wall relative to the affected secondary branch vessel's orifice. In other words, the indented section 230 may not be on the ipsilateral or same side as the orifice of the affected secondary branch vessel 20 (e.g., not in the intersecting portion 14). In this way, the implanted flow diverting apparatus 100's orientation can help divert the inflammation-causing blood components away from the affected secondary branch vessel 20.

In some examples, the indented portion 230 may include pores. In some examples, only the indented portion 230 of the stent frame body 201 includes pores, and the other portion of the stent frame body 201 does not include pores. In other examples, the stent frame body 201 may include the pores in any other suitable portion thereof.

The pores may be provided to filter white blood cells based on the white blood cells' size and/or shape while allowing red blood cells to pass therethrough. In this case, most of the white blood cells from the inlet opening 212 may pass through the outlet opening 222. In some examples, a diameter or a cross-sectional area of the inlet opening 212 may be (substantially) the same as a diameter or a cross-sectional area of the outlet opening 222.

In some examples, the indented portion 230 may be expandable. For example, the stent frame body 201 may be (substantially) in a cylindrical shape when the indented portion 230 is fully expanded.

Other configurations/features/characteristics of the flow diverting apparatus 200 (e.g., inclined angle of the indented portion 230, material of the stent frame body 201, frame wires) may be similar to and/or same as the ones described above with respect to the flow diverting apparatus 100, and, thus, duplicate description may be omitted.

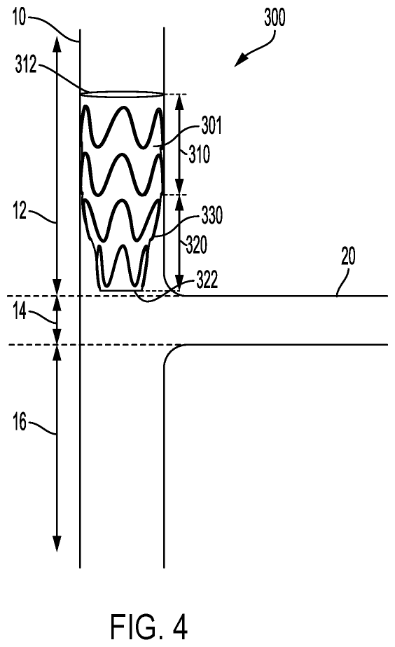

FIG. 4 illustrates another example of a flow diverting apparatus 300 according to an example of the present disclosure. As shown in FIG. 4, the flow diverting apparatus 300 may include a stent frame body 301. The stent frame body 301 may include an inlet section 310 having an inlet opening 312, an outlet section 320 having an outlet opening 322. The outlet section 320 may include a tapered/indented section 330. In some examples, the inlet section 310 may have a cylindrical shape. The flow diverting apparatus 300 may have a wine-bottle shape.

In some examples, the stent frame body 301 may include a cavity extending from the inlet opening 312 to the outlet opening 322. The stent frame body 301 may be configured to pass a flow of fluid from the inlet opening 312 to the outlet opening 322 through the cavity.

In some examples, the inlet opening/outlet opening 312, 322 may have a circular or oval shape. In other examples, the inlet opening/outlet opening 312, 322 may have any other suitable shape.

In some examples, when the flow diverting apparatus 300 is placed in the primary arterial vessel 10, the inlet section 310 of the flow diverting apparatus 300 may be located in the upstream portion 12 of the primary arterial vessel 10.

In some examples, when the flow diverting apparatus 300 is placed in the primary arterial vessel 10, the outlet section 320/indented section 330 of the flow diverting apparatus 300 may be disposed in the upstream portion 12 of the primary arterial vessel 10, as shown in FIG. 4. In other examples, when the flow diverting apparatus 300 is placed in the primary arterial vessel 10, the outlet section 320/indented section 330 of the flow diverting apparatus 300 may be disposed in the intersecting portion 14 or the downstream portion 16 of the primary arterial vessel 10.

In some examples, the indented section 330 may include a curved portion or a concave/convex portion. In some examples, the indented section 230 may include a cross-sectional portion having a shape of crescent, circle, or partial circle (e.g., half-moon). In other examples, the tapered/indented section 230 may include a cross-sectional portion having any other suitable shape (e.g., polygonal shape, regular/irregular shape).

In some examples, the indented portion 330 may include pores. In some examples, only the indented portion 330 of the stent frame body 301 includes pores, and other portion of the stent frame body 301 does not include pores. In other examples, the stent frame body 301 may include the pores in any other suitable portion thereof.

The pores may be provided to filter white blood cells based on the white blood cells' size and/or shape while allowing red blood cells to pass therethrough. In this case, most of the white blood cells from the inlet opening 312 may pass through the outlet opening 322. In some examples, a diameter or a cross-sectional area of the inlet opening 312 may be greater than a diameter or a cross-sectional area of the outlet opening 322.

In some examples, the indented portion 330 may be expandable. For example, the stent frame body 301 may be (substantially) in a cylindrical shape when the indented portion 330 is fully expanded.

Other configurations/features/characteristics of the flow diverting apparatus 300 (e.g., inclined angle of the indented portion 330, material of the stent frame body 301, frame wires, etc.) may be similar to and/or same as the ones described above with respect to the flow diverting apparatus 100 and/or the flow diverting apparatus 200, and, thus, duplicate description may be omitted.

In some examples, a method for treating and/or preventing chronic inflammation affecting regions of the body above the pelvis (e.g., suprapelvic) through the use of an implanted flow diverting apparatus (e.g., the flow diverting apparatus described herein) may be provided. Examples of suprapelvic chronic inflammation that the method and apparatus according to the present disclosure can be used to treat may include, but are not limited to, autoimmune hepatitis, gastritis, inflammatory bowel disease, glomerulonephritis, upper limb osteoarthritis, upper limb rheumatoid arthritis, ankylosing spondylitis, myocarditis, thyroiditis, psoriasis, transplant rejection, and multiple sclerosis.

In some examples, a method of treating and/or preventing inflammatory bowel disease, including Crohn's disease and ulcerative colitis, using a flow diverting apparatus according to the present disclosure may include implanting a first flow diverting apparatus, along the length of the abdominal aorta such that the cylindrical inlet section/first portion of the flow diverting apparatus is positioned above or proximal to the orifice of the celiac trunk. The tapered/indented portion, which can be in a shape of crescent, hour-glass, vortex, or lobular-shaped may extend until it is located adjacent to the celiac trunk orifice. If the tapered distal segment is crescent-shaped, the convex surface of the flow diverting apparatus frame may be oriented (e.g., with the guidance of embedded radiopaque markers) such that it is fixated against the posterior aortic wall.

A second flow diverting apparatus with the same shape as the first flow diverting apparatus may be subsequently deployed within during the same catheter delivery procedure. After the first flow diverting apparatus is delivered to its desired/predetermined position, the catheter delivery system may be pulled distally towards the distal aorta in order to deliver the second flow diverting apparatus. The second flow diverting apparatus may be deployed along the length of the abdominal aorta such that the cylindrical inlet portion/first portion is positioned above or proximal to the orifice of the inferior mesenteric artery. The tapered/indented portion, which can be crescent, hour-glass, vortex, or lobular-shaped, may extend until it is located adjacent to the inferior mesenteric artery orifice. If the tapered distal segment is crescent-shaped, the convex surface of the flow diverting apparatus frame may be oriented (e.g., with the guidance of embedded radiopaque markers) such that it is fixated against the posterior aortic wall. Moreover, the second flow diverting apparatus may, as another option, be cylindrically shaped with no pores and can be delivered at the level of the inferior mesenteric artery orifice to seal off any blood perfusion to the aortic branch vessel.

In some examples, a method of treating and/or preventing inflammatory bowel disease, including Crohn's disease and ulcerative colitis, using a flow diverting apparatus according to the present disclosure may include implanting a flow diverting apparatus according to the present disclosure along the length of the abdominal aorta such that the cylindrical inlet of the flow diverting apparatus is positioned above or proximal to the orifice of the celiac trunk. The tapered/indented portion, which can be crescent, hour-glass, vortex, or lobular-shaped may extend distally and may be located adjacent to the celiac trunk, superior mesenteric artery, and inferior mesenteric artery orifices. If the tapered/indented portion is crescent-shaped, the convex surface of the flow diverting apparatus frame may be oriented (e.g., with the guidance of embedded radiopaque markers) such that it is fixated against the posterior aortic wall. The convex surface may be oriented this way because all the affected secondary branch vessel orifices leading to sites of intestinal inflammation are generally located on the anterior aortic wall. Moreover, the cylindrical outlet section of the flow diverting apparatus may be positioned distal to the orifice of the inferior mesenteric artery within the aorta.

Cases of gastritis and hepatitis may also be treated through a method similar to the one for treating and/or preventing inflammatory bowel disease. In the case of gastritis and hepatitis, however, the tapered/indented portion of the implanted flow diverting apparatus may only extend as distally as the distal edge of the celiac trunk orifice, which is the first major vessel which branches off of the proximal abdominal aorta. Beyond that point, the implanted flow diverting apparatus may or may not include a cylindrical outlet depending on whether the tapered section of the flow diverting apparatus is crescent-shaped. If it is crescent-shaped, then a more distally located cylindrical outlet section may not be necessary. This is because a distal end consisting of a crescent-shaped frame would be fixated against at least 50% of the aortic wall circumference and may thus be unlikely to migrate or become dislodged from its anchor position.

In another example, treatment of multiple sclerosis using a flow diverting apparatus according to the present disclosure may include implanting the flow diverting apparatus along the length of the aortic arch such that the cylindrical inlet of the implanted flow diverting apparatus is positioned proximal to the orifice of the brachiocephalic artery. The tapered/indented portion of the implanted flow diverting apparatus spans the length of the aortic arch and is located adjacent to the brachiocephalic artery, left common carotid artery, and left subclavian artery orifices. If the tapered/indented segment is crescent-shaped, the convex surface of the flow diverting apparatus frame is oriented (e.g., with the guidance of embedded radiopaque markers) such that it is fixated against the inferior aortic wall. The convex surface may be oriented this way because all the affected secondary branch vessel orifices leading to sites of cerebral inflammation are located on the superior aortic arch wall. Lastly, the flow diverting apparatus may or may not include a cylindrical outlet section positioned distal to the left subclavian artery orifice. For instance, deployment of a flow diverting apparatus with a cylindrical outlet distal to the left subclavian artery orifice can help ensure prevention of potential retrograde diastolic flow of leukocytes into aortic arch orifices after they have exited the tapered/indented portion of the apparatus. In some instances, a non-cylindrical outlet design can increase the risk of backward leukocytic flow during diastole after the immune cells had initially exited the tapered/indented section during anterograde systolic flow.

In another example of a suprapelvic chronic inflammation treatment/prevention application for the flow diverting apparatus, treatment/prevention of bilateral upper limb osteoarthritis or left unilateral upper limb osteoarthritis may include implanting a flow diverting apparatus according to the present disclosure along the length of the aortic arch such that the cylindrical inlet section of the implanted flow diverting apparatus would need to be positioned proximal to the orifice of the brachiocephalic artery (in the upstream portion). The tapered/indented portion of the implanted flow diverting apparatus spans the length of the aortic arch and is located adjacent to the brachiocephalic artery, left common carotid artery and left subclavian artery orifices. If the tapered/indented segment is crescent-shaped, the convex surface of the flow diverting apparatus frame is oriented (e.g., with the guidance of embedded radiopaque markers) such that it is fixated against the inferior aortic wall. The convex surface may be oriented this way because all the affected secondary branch vessel orifices leading to sites of upper limb osteoarthritis are located on the superior aortic arch wall. Lastly, the flow diverting apparatus may or may not include a cylindrical outlet section positioned distal to the left subclavian artery orifice. For instance, deployment of a flow diverting apparatus with a cylindrical outlet section distal to the left subclavian artery orifice can help ensure prevention of potential retrograde diastolic flow of leukocytes into aortic arch orifices after they have exited the tapered/indented portion of the apparatus. In some instances, a non-cylindrical outlet design can increase the risk of backward leukocytic flow during diastole after the immune cells had initially exited the tapered section during anterograde systolic flow.

In another example, treatment/prevention of right unilateral upper limb osteoarthritis may include implanting the flow diverting apparatus inlet section such that it is positioned proximal to the brachiocephalic orifice (e.g., in the upstream portion). The tapered middle section of the implanted flow diverting apparatus extends as distally as the distal edge of the brachiocephalic artery orifice. Beyond that point, the flow diverting apparatus may or may not include a cylindrical outlet depending on whether the tapered/indented section of the flow diverting apparatus is crescent-shaped. If it is crescent-shaped, then a more distally located cylindrical outlet may not be incorporated. This is because a crescent-shaped frame of the flow diverting apparatus would be fixated against at least 50% of the aortic wall circumference and would thus be unlikely to migrate or become dislodged from its anchor position.

As mentioned above, with regards to the positioning of the flow diverting apparatus within the primary arterial vessel 10, in some examples, the tapered/indented segment of the flow diverting apparatus (whether the total length of the flow diverting apparatus includes or does not include a cylindrical outlet segment) may be at the same level as the orifice of the affected secondary branch vessel. In an alternative configuration, the flow diverting apparatus may also be positioned within the primary arterial vessel but may be positioned slightly proximal to the level of the affected secondary branch vessel orifice. This alternative configuration may be applied in any vascular setup and may therefore be applied to any of the aforementioned treatment methods related to upper limb osteoarthritis, gastritis, hepatitis, inflammatory bowel disease, or multiple sclerosis.

By having the tapered/indented segment of the flow diverting apparatus more proximal to the level of the affected secondary branch vessel orifice, for example, at a distance equal to or less than the diameter of the affected secondary branch vessel orifice, filtered immune cells may be diverted away from orifice of the affected secondary branch vessel and are unlikely to enter its orifice after exiting the distal end of the flow diverting apparatus.

If the entirety of the flow diverting apparatus is positioned proximal to the orifice of the affected secondary branch vessel, the flow diverting apparatus can be expanded at the tapered/indented segment without causing subsequent obstruction of any part of the target branch vessel lumen should the patient not require further treatment of chronic inflammation. Expansion of the flow diverting apparatus to an entirely cylindrical shape (e.g., with no tapered/indented segment), that is conformant to the cylindrical shape of the primary arterial vessel wall, can be applied to terminate treatment of chronic inflammation because the total volume of white blood cells passing through the primary arterial vessel would no longer be partially or totally obstructed and diverted by the flow diverting apparatus.

In some examples, expansion of the flow diverting apparatus's tapered/indented segment can be achieved by deploying a cylindrical balloon-expandable or self-expandable stent within the already positioned flow diverting apparatus in order to force it open. Another method of expanding the tapered/indented segment of the flow diverting apparatus during termination of chronic inflammation treatment can involve the use of a balloon catheter containing fluid with a high enough temperature in order to cause austenitic structural changes, for example, to the (flow-restricting) stent frame wires (that may be made of or include nitinol) near the indented portion of the flow diverting apparatus so that the frame wires (and the indented portion of the stent frame body associated with the frame wires) are expanded, for example, to have a circular shape.

Although the flow diverting apparatus may be able to filter blood components in order to reduce progression and amplification of inflammatory processes, it is important to consider potential long-term adaptation challenges regarding the porous fabric membrane that may be essential to the device's functionality. Specifically, one of the biggest challenges to long-term usage of a flow diverting apparatus is the risk of endothelialization spreading to the porous portion of the flow diverting apparatus (e.g., in the concave stent-graft surface). Endothelialization can involve the migration or movement of vascular wall cells, such as endothelial cells or smooth muscle cells onto several foreign implant devices including stents. This process may continue until much or all of the device is engulfed and surrounded by the vascular wall that is in contact with the device, especially if the device has open gaps or pores.

Accordingly, endothelialization, in the long-term, may be a major cause of concern for a flow diverting apparatus with pores. This is because the gradual migration of different endothelial wall cells from the stent frame body's proximal surface that is aligned against the vessel wall may progress towards the flow diverting apparatus's more distal concave surface (e.g., indented portion) and eventually cause obstruction and blockage of the pores that are located on the indented portion. The pores of a flow diverting apparatus can be blocked by the endothelialization within less than a month.

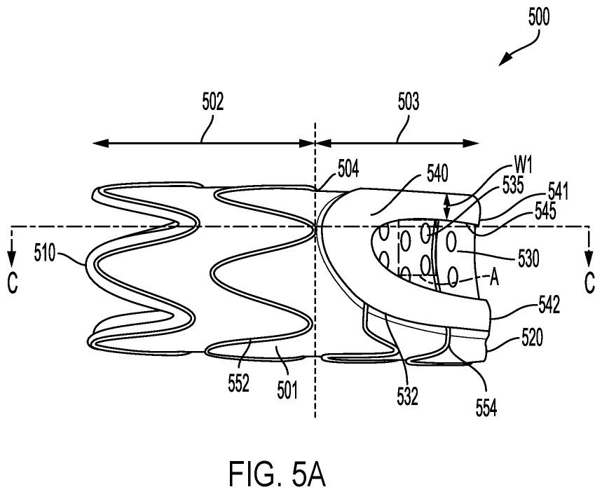

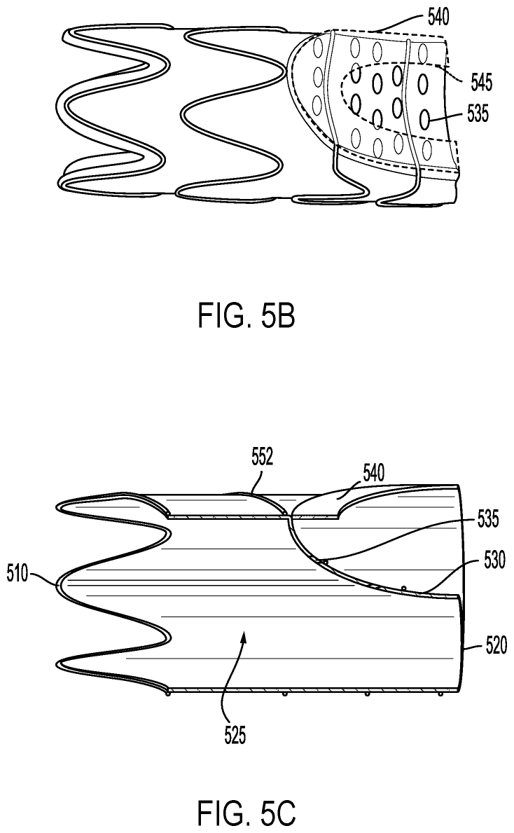

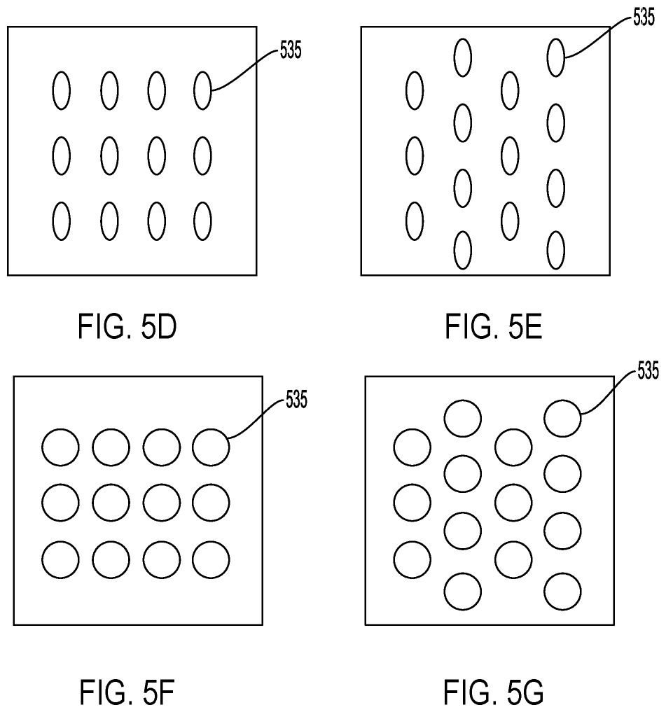

Aspects of the present disclosure may provide a flow diverting apparatus 500 that may address the above-discussed issues of endothelialization. Referring to FIG. 5A-5C, in some examples, the flow diverting apparatus 500 may include a stent frame body 501 and a stent frame extension 540. The stent frame body 501 may include an inlet opening 510, an outlet opening 520, and a cavity 525 extending from the inlet opening 510 to the outlet opening 520. The stent frame body 501 may be configured to pass a flow of fluid from the inlet opening 510 to the outlet opening 520 through the cavity.

In some examples, the inlet opening 510 (e.g., cross-sectional area thereof) may have a circular shape or any other suitable shape. In some examples, the outlet opening 520 (e.g., cross-sectional area thereof) may be in a shape of a circle, partial circle, or crescent. In other examples, the outlet opening 520 may have any other suitable shape.

The stent frame body 501 may include a first portion 502 having the inlet opening 510 and a second portion 503 having the outlet opening 520. In some examples, the first portion 502 may have a cylindrical shape.

In some examples, the stent frame body 501 may include a tapered/indented portion. For example, the second portion 503 of the stent frame body 501 may be tapered from one (cross-sectional) end 504 of the second portion 503 to a first (cross-sectional) location of the second portion 503, thereby forming an indented portion 530. In some examples, the first location of the second portion 503 may be a location on the outlet opening 520, as shown in FIG. 5A. In other examples, the first location may be disposed at any point between the one end 504 and the outlet opening 520. In some examples, the second portion 503 may be defined as a portion of the stent frame body 501 that extends from a (cross-sectional) point where the tapering starts to a point where the outlet opening 520 is disposed, and the first portion 502 is the rest of the portion of the stent frame body 501 that is not the second portion 503.

In some examples, the indented portion 530 may include pores 535. In some examples, only the indented portion 530 of the stent frame body 501 includes pores 535, and the other portion of the stent frame body 501 does not include pores. In other examples, the stent frame body 501 may include the pores 535 in any other suitable portion thereof.

The pores may be provided to filter white blood cells based on the white blood cells' size and/or shape while allowing red blood cells to pass therethrough. In this case, most (e.g., over 90%, 95%, 98%, or 99%) of the white blood cells from the inlet opening 510 may pass through the outlet opening 520. In some examples, a diameter or a cross-sectional area of the inlet opening 510 may be greater than a diameter or a cross-sectional area of the outlet opening 520.

In some examples, the indented portion 530 may be expandable. For example, the stent frame body 501 may be (substantially) in a cylindrical shape when the indented portion 530 is fully expanded.

In some examples, the indented portion 530 may include a curved portion, a concave/convex portion. In some examples, the tapered portion of the flow diverting apparatus 500 may take the form of a vortex, cone, hourglass, lobular, or a crescent shape. In some examples, the tapered/indented portion may include a cross-sectional portion having a shape of crescent, circle, or partial circle (e.g., half-moon). In other examples, the tapered/indented portion may include a cross-sectional portion having any other suitable shape (e.g., polygonal shape, regular/irregular shape).

Other configurations/features/characteristics of the flow diverting apparatus 500 (e.g., inclined angle of the indented portion 530, material of the stent frame body 501, frame wires 552, 554) may be similar to and/or same as the ones described above with respect to the flow diverting apparatus 100, and, thus, duplicate description may be omitted.

In some examples, as shown in FIG. 5A, the stent frame extension 540 may protrude from at least a portion of a side surface of the stent frame body 501 and protrude away from the inlet opening 510. For example, in some examples, the stent frame extension 540 may protrude from the edge 532 of the indented portion 530. In this case, at least a portion of the stent frame extension 540 may protrude from the one end 504 of the second portion 503 away from the inlet opening 510. In other examples, the stent frame extension 540 may protrude from any other suitable portion of the surface of the stent frame body 501 (e.g., between the inlet opening 510 and the edge 532 of the indented portion 530).

In some examples, as shown in FIG. 5A, the stent frame extension 540 may be in a U shape. In this case, in some examples, the stent frame extension 540 may have the same width W1 (e.g., a distance between the edge 532 of the indented portion 530 to a free end 545 of the stent frame extension 540) from a first side end 541 to a second side end 542. In other examples, the width W1 of the stent frame extension 540 may vary between the first side end 541 and the second side end 542.

The stent frame extension 540 is configured to be disposed between at least a portion of the indented portion 530 and a vessel wall (e.g., vessel wall of the primary arterial vessel 10), thereby preventing a migration of vessel wall cells onto the indented portion.

FIGS. 5D-5G illustrate example shapes and arrangements of the pores 535, for example, taken from area A of the flow diverting apparatus 500. As shown in FIGS. 5D and 5E, in some examples, the pores 535 may have an oval shape. In this case, the size of the pores 535 may be about 4 to about 36 μm in length and about 8 to about 64 μm in width, for example, about 4 to about 9 μm in length and about 8 to about 16 μm in width, about 9 to about 18 μm in length and about 16 to about 32 μm in width, about 18 to about 27 μm in length and about 32 to about 48 μm in width, or about 27 to about 36 μm in length and about 48 to about 64 μm in width. In some examples, the oval pores 535 may be oriented perpendicular or parallel to the direction of blood flow (e.g., a longitudinal axis direction extending from the inlet opening 510 to the outlet opening 520).

As shown in FIGS. 5F and 5G, in some examples, the pores 535 may have a circular shape. In this case, the pores 535 may have a diameter in a range of about 8 μm to about 70 μm, for example, about 10 μm to about 50 μm, about 20 μm to about 40 μm, about 25 μm to about 35 μm, or about 8 μm to about 15 μm.

In other examples, the pores 535 may have any other suitable shape, such as triangle, rectangular, square, hexagon, any other polygonal shape (with or without rounded corners), or regular/irregular shape. In this case, the pores 535 may have a length (e.g., any longest straight line distance between two points on the edge of the pores 535) in a range of about 8 μm to about 70 μm, for example, about 10 μm to about 50 μm, about 20 μm to about 40 μm, about 25 μm to about 35 μm, or about 8 μm to about 15 μm.

In some examples, as shown in FIGS. 5D and 5F, the pores 535 are arranged to form a plurality of columns and rows in a vertical and horizontal direction, respectively. In each column and row, the pores 535 are spaced apart from each other at the same distance. In this case, four adjacent pores 535 (e.g., two adjacent pores 535 from a first column and two adjacent pores 535 from a second column that are disposed right next to the two adjacent pores 535 from the first column) may form a square or rectangular shape.

In some examples, as shown in FIGS. 5E and 5G, the pores 535 are arranged to form a plurality of columns in a vertical direction. In each column, the pores 535 are spaced apart from each other at the same distance. The pores 535 in 2N−1 columns (N is an integer equal to or greater than 0) are arranged to form a first group of rows in a horizontal direction, and the pores 535 in 2N columns are arranged to form a second group of rows in the horizontal direction. In this case, four adjacent pores 535 (e.g., two adjacent pores 535 from a first column and two adjacent pores 535 from a second column that are disposed right next to the two adjacent pores 535 from the first column) may form a parallelogram shape. In some examples, four adjacent pores 535 in the 2N−1 columns (e.g., two adjacent pores 535 from a first column and two adjacent pores 535 from a third column that are disposed right next to the two adjacent pores 535 from the first column) may form a virtual square/rectangular 537, and a pore 535 in the 2N columns may be disposed in a center of the virtual square/rectangular 537.

In other examples, the pores 535 may be arranged in any other suitable way (e.g., forming a hexagonal, pentagonal, or any other polygonal shape, or arranged randomly). In some examples, the pores 535 may be homogenous in shape throughout the indented portion 530/stent frame body 501. In other examples, the pores 535 may have different pore shapes within the indented portion 530/stent frame body 501.

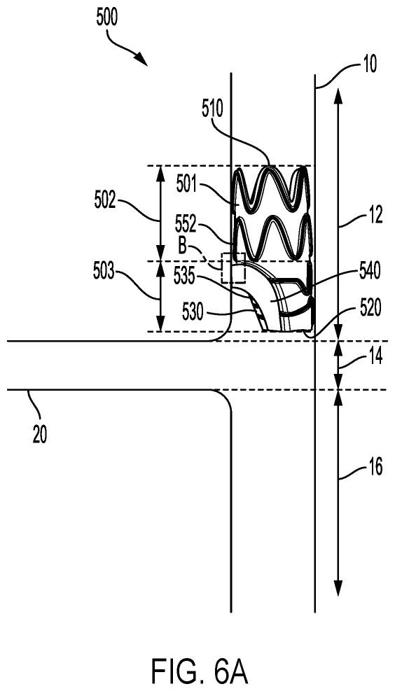

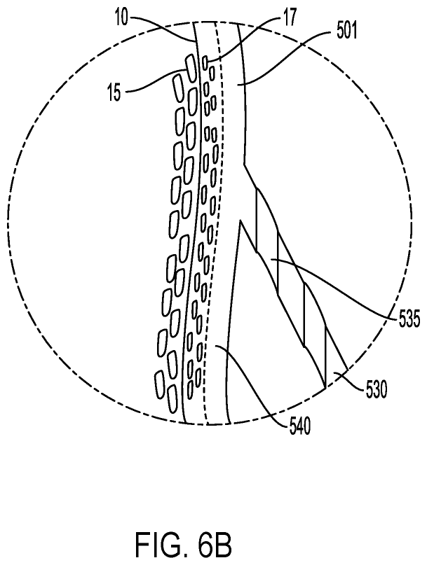

As shown in FIG. 6A, the flow diverting apparatus 500 may be placed within a primary arterial vessel 10 such that the inlet opening 510 of the flow diverting apparatus 100 (and the first portion 502) may be proximal to (upstream) the orifice of the affected secondary branch vessel 20 (e.g., in the upstream portion 12).

In some examples, when the flow diverting apparatus 500 is placed in the primary arterial vessel 10, the indented portion 530 and/or the outlet opening 520 of the stent frame body 501 may be positioned in the upstream portion 12 of the primary arterial vessel 10. In other examples, when the flow diverting apparatus 500 is placed in the primary arterial vessel 10, the indented portion 530 (at least a portion thereof) and/or the outlet opening 520 of the stent frame body 501 may be disposed in the intersecting portion 14 and/or the downstream portion 16 of the primary arterial vessel 10.

In some examples, the stent frame body 501 and/or the stent frame extension 540 may be configured to have a thickness that is sufficient to prevent the endothelialization. In some examples, a thickness of the stent frame body 501 and/or the stent frame extension 540 may be at least 40 μm, for example, at least 50 μm, at least 60 μm, at least 70 μm, or at least 80 μm. Endothelial cellular components may not be able to migrate through a wall having a thickness of more than 40 μm and/or having an internodal distance of at least 30 μm. Therefore, migration of endothelial cellular components onto the flow/luminal surface of the flow diverting apparatus may be prevented through this route.

In case of PET fabric usage, the total thickness of PET may have a total thickness of at least 20 μm in order to prevent endothelial migration through the thickness of the stent frame body 501 onto the flow surface. Spreading of endothelial cellular components may not occur by crossing over a proximal or distal end of the stent frame body from the stent frame body's exterior wall surface to the stent frame body's interior flow/luminal surface.

FIG. 6B is an expanded and cross-sectional view of the area B of FIG. 6A. As discussed above, one of the biggest challenges to long-term usage of a flow diverting apparatus is the risk of endothelialization spreading to the pores of the flow diverting apparatus (e.g., in the tapered/indented portion). For example, as shown in FIG. 6B, after the installation of the flow diverting apparatus, the vascular wall cells 15, 17, such as endothelial cells or smooth muscle cells, can penetrate into and/or move along the surface of the flow diverting apparatus, which can block the pores in the indented portion. Aspects of the present disclosure may prevent the migration of the endothelial wall cells and the blockage of the pores by these endothelial wall cells, for example, by providing the stent frame extension 540, which may be disposed between the vessel wall and the indented portion 530. Also, when the stent frame body 501 and/or the stent frame extension 540 have a thickness in the above-discussed range, the endothelial wall cells would not be able to pass through the thickness of the stent frame body 501 and/or the stent frame extension 540, thereby, preventing the vessel wall cells from blocking the pores of the indented portion 530.

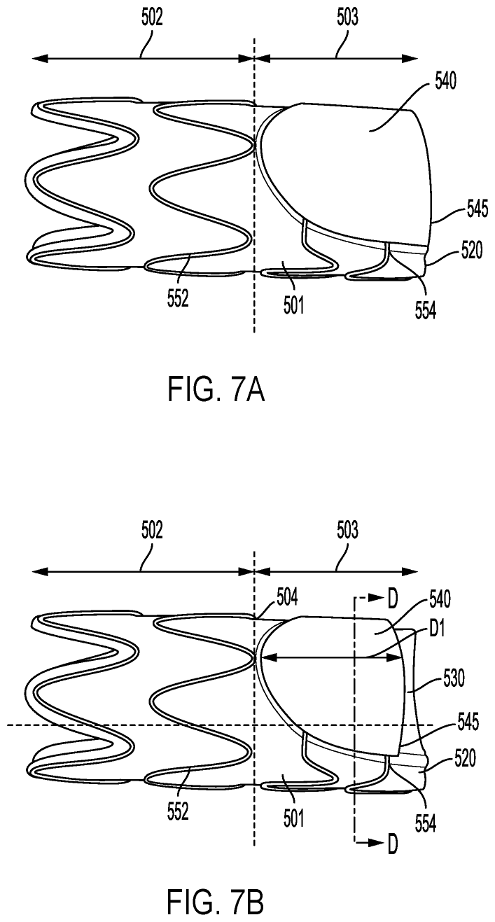

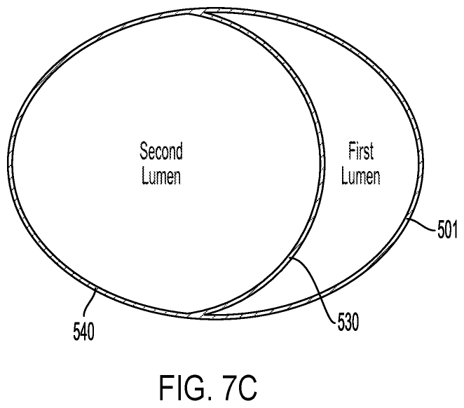

In FIGS. 7A and 7B, the stent frame extension 540 has a shape different from the one in FIG. 5A. In FIG. 7A, the stent frame extension 540 may cover all area of the indented portion 530. In this case, a portion of the stent frame extension 540 may be in direct contact with the outlet opening 520. In some examples, the second portion 503, together with the stent frame extension 540, may have a cylindrical shape.

In FIG. 7B, the stent frame extension 540 may partially cover the indented portion 530. For example, the stent frame extension 540 may cover the indented portion 530 from the one end 504 of the first portion (where the tapering starts) to a point between the one end 504 and the outlet opening 520. In this case, the stent frame extension 540 may not be in direct contact with the outlet opening 520.

In some examples, a shortest distance D1 between a point of the stent frame extension 540 on the one end 504 and a point of the free end 545 of the stent frame extension 540 may be in a range of about 2 mm to about 20 mm, for example, about 2 mm to about 5 mm, about 5 mm to about 15 mm, or about 15 mm to about 20 mm.

In some examples, the stent frame extension 540 may not include any frame wires. In other examples, the stent frame extension 540 may include one or more frame wires (similar to the frame wires 152, 154 discussed above) extending around a perimeter of the stent frame extension 540. In some examples, when the stent frame extension 540 includes one or more frame wires, the indented portion 530 of the stent frame body 501 may not include any stent frame wires. That is, a stent wire support may only exist around the circular circumference of the second portion 503 of the stent frame body 501 that is completely aligned with the primary arterial vessel wall and around the stent frame extension 540. This stent wire may therefore resemble the structure and shape of stent wires 542 located near inlet opening 510.

FIG. 7C is a cross-sectional view of the flow diverting apparatus 500 of FIG. 7B taken along line D-D in FIG. 7B. As shown in FIG. 7C, the stent frame body 501 (in the second portion 503) may form a first lumen, and the stent frame extension 540 and the indented portion 530 may form a second lumen. In some examples, the first lumen may allow unfiltered blood (e.g., large white blood cells, such as neutrophils and macrophages) to flow therethrough, and the second lumen may allow blood that has been filtered through the pores 535 to flow therethrough.

In some examples, the first lumen may be in a crescent shape. In other examples, the first lumen may have any other suitable shape (e.g., circle, partial circle, oval). In some examples, the second lumen may be in a shape of a circle, partial circle, or oval. In other examples, the second lumen may have any other suitable shape.

In some examples, as shown in FIG. 6B, the implanted flow diverting apparatus 500 may be oriented such that the outlet opening 520 is located on the opposite side of the vessel lumen relative to where the orifice of the affected secondary branch vessel 20 is located. In this case, the indented portion 530 may face in a direction in which the affected secondary branch vessel 20 extends from the orifice.

As shown above, the stent frame extension 540 may provide a structural support to the stent frame body 501 in the second portion 503. For example, while in the flow diverting apparatus 100 of FIG. 1, the structural support for the second portion 103 is mainly provided by the stent frame wires 154. However, in the flow diverting apparatus 500 of FIGS. 5A, 7A, and/or 7B, in addition to the stent wires 554 extending around the perimeter of the second portion 503, the structural support for the second portion 503 is additionally provided by the stent frame extension 540, where the second lumen (and/or the first and second lumen together) may have a circular/oval ring shape, which is similar to the shape of the inlet opening 510 or the stent wires near the inlet opening 510.

In some examples, the stent frame extension 540 may be provided to other flow diverting apparatus designs (e.g., the ones illustrated in FIGS. 3 and 4). The configurations/features/characteristics of the stent frame extension that is applied to these other flow diverting apparatus designs may be similar to and/or same as the ones described above with respect to the flow diverting apparatus 500. For example, the stent frame extension may protrude from at least a portion of a side surface of the stent frame body 201/301 and protrude away from the inlet opening 210/310. In some examples, the stent frame extension may protrude from the edge of the indented portion 230/330. In other examples, the stent frame extension may protrude from any other suitable portion of the surface of the stent frame body 201/301 (e.g., between the inlet opening 212/312 and the edge of the indented portion 230/330). The stent frame extension is configured to be disposed between at least a portion of the indented portion 230/330 and a vessel wall (e.g., vessel wall of the primary arterial vessel 10), thereby preventing a migration of vessel wall cells onto the indented portion.

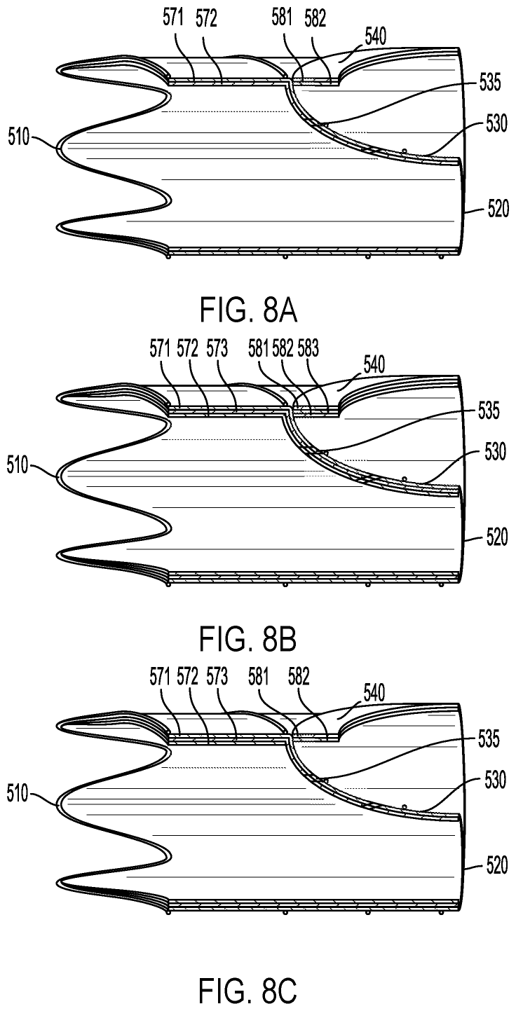

In some examples, a flow diverting apparatus according to the present disclosure may be made with a material that may be composed of two or three parallel layers. For example, referring to FIG. 8A, the stent frame body 501 and the stent frame extension 540 may have a bilayer configuration by including two parallel layers—an exterior layer 571/581 and an interior layer 572/582. In some examples, the exterior layer 571/581 may be made of or include PET and the interior layer 572/582 may be made of or include ePTFE. In other examples, the exterior and interior layers may include any other suitable materials.

Referring to FIG. 8B, in some examples, the stent frame body 501 and the stent frame extension 540 may have a trilayer configuration by including three parallel layers—an exterior layer 571/581, a middle layer 573/583, and an interior layer 572/582. In some examples, the exterior layer 571/581 and interior layer 572/582 may be made of or include ePTFE and the middle layer 573/583 may be made of or include PET. In other examples, the exterior, middle, and interior layers may be made of or include any other suitable materials.

Referring to FIG. 8C, in some examples, a flow diverting apparatus according to the present disclosure may be made with a material having a hybrid configuration. For example, as shown in FIG. 8C, the stent frame body 501 (and the stent frame extension 540) may have a trilayer configuration except for the indented portion 530, which may have a single or bilayer configuration. Non-limiting examples of the material for the stent frame body 501 and the stent frame extension 540 (e.g., for the exterior/middle/interior layer) may include ePTFE, PET, urethane-based material, or any combination thereof.

In some examples, a method for treating and/or preventing chronic inflammation or lymphedema affecting regions of the body below the pelvis (e.g., infrapelvic) p through the use of an implanted flow diverting apparatus according to the present disclosure (e.g., the flow diverting apparatus described herein) may be provided. With regard to cases of unilateral or bilateral lower limb chronic inflammation (e.g., osteoarthritis of the knee or hip), the flow diverting apparatus may be implanted such that it is positioned at the level of the infrarenal aorta above the aortic bifurcation. In some examples, the most tapered/indented portion of the flow diverting apparatus, also known as its throat, may be anchored to the aortic wall within a range of 0.5 to 7.0 cm proximal (upstream) to the aortic bifurcation. The aim of implanting the flow diverting apparatus particularly within the infrarenal aorta may be to divert migration of inflammatory immune cells towards the less-affected, or otherwise non-affected, lower limb. By favoring passage of inflammatory cells, such as monocytes, neutrophils, macrophages, and granulocytes, towards the healthier contralateral lower limb and away from the arterial system of the diseased lower limb, overt inflammation causing severe pain and discomfort can be mechanically modulated. Furthermore, given that the level of capillary bed permeability in the healthier contralateral limb may be lower than that of the diseased limb due to less inflammatory signaling, the probability of complications arising due to shunting of leukocytes to the arterial system of the healthy limb may be reduced.

In some examples, a flow diverting apparatus having a crescent-shaped tapered section may be used to achieve shifting of leukocytic passage unilaterally. One benefit of using the crescent-shaped throat is that the orientation of the crescent-shaped tapered section is adaptable and is not forced to assume a centerlined and symmetrical position along the aorta each time. As such, in various examples, the flow diverting apparatus can be implanted (e.g., with the guidance of strategically placed radiopaque markers located on the apparatus) such that the midline of the most narrowed portion of the crescent-shaped tapered section is rotated within a range of 10 to 90 degrees from the anteroposterior midline of the aorta.

In at least some aspects, in order to prevent redistribution of leukocytes throughout the cross-sectional area of the aortic lumen following exit from the distal end (downstream opening) of the flow diverting apparatus, the throat of the flow diverting apparatus can be the distal end of the flow diverting apparatus and can be positioned within close vertical distance (with the patient standing) of the iliac artery supplying the healthier lower limb. Orienting the crescent-shaped throat, which may be designed as the distal end, in this way may help create a jet stream of leukocytic flow and secure its passage into the arterial system of the healthier lower limb.

With regard to treating unilateral lymphedema of the lower limb, a flow diverting apparatus according to the present disclosure can be implanted at the level of the infrarenal aorta above the aortic bifurcation where the infrarenal aorta splits into the right and left common iliac arteries (which may be referred to as secondary branches). This positioning of the flow diverting apparatus may help direct total blood flow towards the healthy contralateral lower limb as opposed to the diseased limb. By partially reducing the arterial pressure of the lymphedematous limb, collection of trapped lymphatic fluid can be moderated, leading to reduced circumferential swelling and potentially better function.

Similarly, the flow diverting apparatus can also be used as a treatment method for unilateral lower limb lymphedema patients following lymphovenous bypass surgery of the affected lower limb. As the lymphovenous bypass surgical procedure involves surgical anastomosis of lymphatic vessels to the venous vascular system, rerouting of lymphatic fluid towards veins is achieved for restored drainage. However, this procedure may be limited in efficacy. Thus, parallel and synergetic treatment methods that can significantly enhance clinical outcomes seen in post-lymphovenous bypass patients may be needed.

By partially diverting total blood flow away from the diseased lower limb in these post-surgical patients using an implanted flow diverting apparatus as described above, arterial pressure may be reduced and thus venous return from the affected limb would also consequentially be decreased. This critical reduction of venous blood pressure would allow for enhanced drainage of excess lymph fluid via lymphatic vessels that have been anastomosed to venous vessels. This is because the anastomosed veins receiving excess lymphatic fluid have residual volume capacity to transport more circulating bodily fluid if necessary.

In some aspects, the use of the flow diverting apparatus is not limited to the duration of patient treatment but can also be relevant following the end of flow diversion therapy, for example, in patients suffering from unilateral lower limb chronic inflammation, unilateral lower limb lymphedema or congestive heart failure. If the end of flow diversion therapy is required, complete blood flow patency can be restored by expanding the tapered or narrowed segment of the flow diversion apparatus, such that its total stent frame perimeter becomes aligned with most or all of the aortic endothelial lining. This can be done by deploying an additional, secondary balloon-expandable or self-expanding stent, which has a uniformly conventional, cylindrical shape from within the primary tapered stent device. By designing the secondary conventional stent such that the wire diameters are larger than those of the primary tapered stent, the radial force exerted by the secondary conventional stent on the luminal side of the primary tapered stent may overcome resistance to expand and may ultimately restore normal vessel patency.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

### EMBODIMENTS

Various aspects of the subject matter described herein are set out in the following numbered embodiments:

Embodiment 1. A flow diverting apparatus comprises a stent frame body; and
a stent frame extension, wherein the stent frame body comprises: an inlet opening: an outlet opening; and a cavity extending from the inlet opening to the outlet opening, wherein the stent frame body is configured to pass a flow of fluid from the inlet opening to the outlet opening through the cavity, wherein the stent frame body includes a first portion having the inlet opening, a second portion having the outlet opening, wherein the second portion is tapered from one end of the second portion to a first location of the second portion, thereby forming an indented portion having a plurality of pores, wherein the first location of the second portion is disposed between the one end and the outlet opening or on the outlet opening, wherein the pores of the indented portion are configured to filter white blood cells based on the white blood cells' size and/or shape while allowing red blood cells to pass therethrough, wherein a cross-sectional area of the inlet opening is greater than a cross-sectional area of the outlet opening, wherein the stent frame extension protrudes from at least a portion of a side surface of the stent frame body and protrudes away from the inlet opening, wherein the stent frame extension is configured to be disposed between at least a portion of the indented portion and a vessel wall, thereby preventing a migration of vessel wall cells onto the indented portion.

Embodiment 2. The flow diverting apparatus of embodiment 1, wherein the plurality of pores of the indented portion are in an oval shape.

Embodiment 3. The flow diverting apparatus of any one of embodiments 2, wherein a length of the pores of the indented portion are in a range of about 4 μm to about 36 μm and a width of the pores of the indented portion are in a range of about 8 μm to about 64 μm.

Embodiment 4. The flow diverting apparatus of embodiment 1, wherein the plurality of pores of the indented portion are in a circular shape.

Embodiment 5. The flow diverting apparatus of embodiment 4, wherein a diameter of the pores of the indented portion are in a range of about 8 μm to about 70 μm.

Embodiment 6. The flow diverting apparatus of any one of embodiments 1-5, wherein a thickness of the stent frame body and/or the stent frame extension is at least 40 μm.

Embodiment 7. The flow diverting apparatus of any one of embodiments 1-6, wherein the indented portion is expandable.

Embodiment 8. The flow diverting apparatus of embodiment 7, wherein the stent frame is in a cylindrical shape when the indented portion is fully expanded.

Embodiment 9. The flow diverting apparatus of any one of embodiments 1-8, wherein the indented portion is curved.

Embodiment 10. The flow diverting apparatus of any one of embodiments 1-8, wherein the stent frame body defines a longitudinal axis extending from the inlet opening to the outlet opening, wherein an angle formed between the longitudinal axis and the indented portion is in a range of about 10 degrees to about 90 degrees.

Embodiment 11. The flow diverting apparatus of any one of embodiments 1-10, wherein the stent frame body is made with at least one of polyester, polytetrafluoroethylene, expanded polytetrafluoroethylene, and polyethylene terephthalate.

Embodiment 12. The flow diverting apparatus of any one of embodiments 1-11, wherein the stent frame body and/or the stent frame extension comprises an exterior layer and an interior layer.

Embodiment 13. The flow diverting apparatus of embodiment 12, wherein the exterior layer comprises polyethylene terephthalate and the interior layer comprises expanded polytetrafluoroethylene.

Embodiment 14. The flow diverting apparatus of embodiment 12, wherein the stent frame body and/or the stent frame extension further comprises a middle layer between the exterior layer and the interior layer.

Embodiment 15. The flow diverting apparatus of embodiment 14, wherein the exterior and interior layers comprise expanded polytetrafluoroethylene and the middle layer comprises polyethylene terephthalate.

Embodiment 16. The flow diverting apparatus of any one of embodiments 1, further comprises one or more frame wires extending around a perimeter of the stent frame body.

Embodiment 17. The flow diverting apparatus of any one of embodiment 1-16, wherein at least a portion of the stent frame extension protrudes from the one end of the second portion away from the inlet opening.

Embodiment 18. A method of treating and/or preventing chronic inflammation in a patient comprises: implanting a flow diverting apparatus in a primary arterial vessel of a patient, the flow diverting apparatus including an inlet opening and an outlet opening, wherein the flow diverting apparatus is implanted such that the inlet opening is positioned within the primary arterial vessel upstream an orifice of a secondary branch vessel that branches off of the primary arterial vessel and the outlet opening is positioned at, upstream, or downstream the orifice, wherein the implanted flow diverting apparatus is positioned and configured such that a proportion of inflammation-causing components in blood of the patient in the primary arterial vessel upstream the flow diverting apparatus being directed away from the secondary branch vessel is greater than a proportion of the inflammation-causing components in the blood of the patient in the primary arterial vessel upstream the flow diverting apparatus being directed towards the secondary branch vessel, and wherein the secondary branch vessel leads to a portion of a body of the patient affected with chronic inflammation.

Embodiment 19. The method of embodiment 18, wherein the inflammation-causing components of the blood include neutrophils, monocytes, granulocytes, macrophages, lymphocytes, dendritic cells, mast cells, and plasma cells.

Embodiment 20. The method of any one of embodiments 18-19, wherein the secondary branch vessel is a celiac trunk of the patient, and wherein the flow diverting apparatus is implanted along a length of an abdominal aorta such that the inlet opening of the flow diverting apparatus is positioned upstream an orifice of the celiac trunk.

Embodiment 21. The method of any one of embodiments 18-20, wherein the secondary branch vessel is a brachiocephalic artery of the patient, and wherein the flow diverting apparatus is implanted along a length of an aortic arch such that the inlet opening of the flow diverting apparatus is positioned upstream an orifice of the brachiocephalic artery.

Embodiment 22. The method of any one of embodiments 18-21, wherein the chronic inflammation comprises at least one of autoimmune hepatitis, gastritis, inflammatory bowel disease, glomerulonephritis, upper limb osteoarthritis, upper limb rheumatoid arthritis, ankylosing spondylitis, myocarditis, psoriasis, transplant rejection, and multiple sclerosis.

Embodiment 23. The method of any one of embodiments 18-22, wherein the implanted flow diverting apparatus is positioned and configured such that a proportion of the inflammation-causing components of the blood of the patient in the primary arterial vessel upstream the flow diverting apparatus being directed to the primary arterial vessel downstream the flow diverting apparatus is greater than a proportion of the inflammation-causing components of the blood of the patient in the primary arterial vessel upstream the flow diverting apparatus being directed to the secondary branch vessel.

Embodiment 24. The method of any one of embodiments 18-23, wherein the secondary branch vessel is a first secondary branch vessel, wherein the implanted flow diverting apparatus is positioned and configured such that a proportion of the inflammation-causing components of the blood of the patient in the primary arterial vessel upstream the flow diverting apparatus being directed to a second secondary branch vessel is greater than a proportion of the inflammation-causing components of the blood of the patient in the primary arterial vessel upstream the flow diverting apparatus being directed to the first secondary branch vessel, and wherein the second secondary branch vessel branches off of the primary arterial vessel and leads to a portion of the body of the patient less affected with the chronic inflammation than the portion of the body to which the first secondary branch leads.

Embodiment 25. The method of any one of embodiments 18-24, wherein the inflammation-causing components of the blood are cells having a diameter equal to or greater than 8 μm.

Embodiment 26. The method of any one of embodiments 18-25, wherein the flow diverting apparatus is constructed of a mesh material having a pore diameter size within a range of 8 to 70 μm.

Embodiment 27. The method of any one of embodiments 18-25, wherein the flow diverting apparatus is constructed of a mesh material having a pore length in a range of about 4 μm to about 36 μm and a pore width in a range of about 8 μm to about 64 μm.

Embodiment 28. The method of any one of embodiments 18-27, wherein a portion of the flow diverting apparatus tapers from a first cross section to a second cross section at an angle within a range of 10 to 90 degrees, wherein the angle is measured from an axis extending through a length of the flow diverting apparatus.

Embodiment 29. A method of treating and/or preventing lymphedema or chronic inflammation in a patient comprises: implanting a flow diverting apparatus in an infrarenal aorta of the patient such that the flow diverting apparatus is positioned upstream orifices of right and left common iliac arteries, wherein the implanted flow diverting apparatus is positioned and configured such that a greater proportion of blood or a greater proportion of inflammation-causing components of the blood of the patient, is directed from the infrarenal aorta to the orifice of the right common iliac artery or the left common iliac artery as compared to the orifice of the other of the right common iliac artery or the left common iliac artery, and wherein the other of the right common iliac artery or the left common iliac artery leads to a lower limb of the patient affected with lymphedema or chronic inflammation.

Embodiment 30. The method of embodiment 29, wherein the flow diverting apparatus is implanted subsequent to lymphovenous bypass surgery of the lower limb affected with the lymphedema.

Embodiment 31. The method of any one of embodiments 29-30, wherein the implanted flow diverting apparatus is positioned in the infrarenal aorta such that a most tapered section of the flow diverting apparatus is upstream an aortic bifurcation of the patient by a distance within a range of about 0.5 cm to about 7.0 cm.

Embodiment 32. The method of any one of embodiments 29-31, wherein the flow diverting apparatus includes a crescent-shaped tapered portion, and wherein the implanted flow diverting apparatus is positioned such that a midline of the crescent-shaped tapered portion is rotated 10 to 90 degrees with respect to an anteroposterior midline of the infrarenal aorta.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

1. A flow diverting apparatus comprising:
a stent frame body; and
a stent frame extension,
wherein the stent frame body comprises:
an inlet opening;
an outlet opening; and
a cavity extending from the inlet opening to the outlet opening, wherein the stent frame body is configured to pass a flow of fluid from the inlet opening to the outlet opening through the cavity,
wherein the stent frame body includes a first portion having the inlet opening, a second portion having the outlet opening,
wherein the second portion is tapered from one end of the second portion to a first location of the second portion, thereby forming an indented portion having a plurality of pores, wherein the first location of the second portion is disposed between the one end and the outlet opening or on the outlet opening,
wherein the pores of the indented portion are configured to filter white blood cells based on the white blood cells' size and/or shape while allowing red blood cells to pass therethrough,
wherein a cross-sectional area of the inlet opening is greater than a cross-sectional area of the outlet opening,
wherein the stent frame extension protrudes from at least a portion of a side surface of the stent frame body and protrudes away from the inlet opening,
wherein the stent frame extension is configured to be disposed between at least a portion of the indented portion and a vessel wall, thereby preventing a migration of vessel wall cells onto the indented portion.

2. The flow diverting apparatus of claim 1, wherein the plurality of pores of the indented portion are in an oval shape.

3. The flow diverting apparatus of claim 2, wherein a length of the pores of the indented portion are in a range of about 4 μm to about 36 μm and a width of the pores of the indented portion are in a range of about 8 μm to about 64 μm.

4. The flow diverting apparatus of claim 1, wherein the plurality of pores of the indented portion are in a circular shape.

5. The flow diverting apparatus of claim 4, wherein a diameter of the pores of the indented portion are in a range of about 8 μm to about 70 μm.

6. The flow diverting apparatus of claim 1, wherein a thickness of the stent frame body and/or the stent frame extension is at least 40 μm.

7. The flow diverting apparatus of claim 1, wherein the indented portion is expandable.

8. The flow diverting apparatus of claim 7, wherein the stent frame is in a cylindrical shape when the indented portion is fully expanded.

9. The flow diverting apparatus of claim 1, wherein the indented portion is curved.

10. The flow diverting apparatus of claim 1, wherein the stent frame body defines a longitudinal axis extending from the inlet opening to the outlet opening, wherein an angle formed between the longitudinal axis and the indented portion is in a range of about 10 degrees to about 90 degrees.

11. The flow diverting apparatus of claim 1, wherein the stent frame body is made with at least one of polyester, polytetrafluoroethylene, expanded polytetrafluoroethylene, and polyethylene terephthalate.

12. The flow diverting apparatus of claim 1, wherein the stent frame body and/or the stent frame extension comprises an exterior layer and an interior layer.

13. The flow diverting apparatus of claim 12, wherein the exterior layer comprises polyethylene terephthalate and the interior layer comprises expanded polytetrafluoroethylene.

14. The flow diverting apparatus of claim 12, wherein the stent frame body and/or the stent frame extension further comprises a middle layer between the exterior layer and the interior layer.

15. The flow diverting apparatus of claim 14, wherein the exterior and interior layers comprise expanded polytetrafluoroethylene and the middle layer comprises polyethylene terephthalate.

16. The flow diverting apparatus of claim 1, further comprises one or more frame wires extending around a perimeter of the stent frame body.

17. The flow diverting apparatus of claim 1, wherein at least a portion of the stent frame extension protrudes from the one end of the second portion away from the inlet opening.

18. A method of treating and/or preventing chronic inflammation in a patient comprising:
implanting a flow diverting apparatus in a primary arterial vessel of a patient, the flow diverting apparatus including an inlet opening and an outlet opening, wherein the flow diverting apparatus is implanted such that the inlet opening is positioned within the primary arterial vessel upstream an orifice of a secondary branch vessel that branches off of the primary arterial vessel and the outlet opening is positioned at, upstream, or downstream the orifice,
wherein the implanted flow diverting apparatus is positioned and configured such that a proportion of inflammation-causing components in blood of the patient in the primary arterial vessel upstream the flow diverting apparatus being directed away from the secondary branch vessel is greater than a proportion of the inflammation-causing components in the blood of the patient in the primary arterial vessel upstream the flow diverting apparatus being directed towards the secondary branch vessel, and
wherein the secondary branch vessel leads to a portion of a body of the patient affected with chronic inflammation.

19. The method of claim 18, wherein the inflammation-causing components of the blood include neutrophils, monocytes, granulocytes, macrophages, lymphocytes, dendritic cells, mast cells, and plasma cells.

20. The method of claim 18, wherein the secondary branch vessel is a celiac trunk of the patient, and wherein the flow diverting apparatus is implanted along a length of an abdominal aorta such that the inlet opening of the flow diverting apparatus is positioned upstream an orifice of the celiac trunk.

21. The method of claim 18, wherein the secondary branch vessel is a brachiocephalic artery of the patient, and wherein the flow diverting apparatus is implanted along a length of an aortic arch such that the inlet opening of the flow diverting apparatus is positioned upstream an orifice of the brachiocephalic artery.

22. The method of claim 18, wherein the chronic inflammation comprises at least one of autoimmune hepatitis, gastritis, inflammatory bowel disease, glomerulonephritis, upper limb osteoarthritis, upper limb rheumatoid arthritis, ankylosing spondylitis, myocarditis, psoriasis, transplant rejection, and multiple sclerosis.

23. The method of claim 18, wherein the implanted flow diverting apparatus is positioned and configured such that a proportion of the inflammation-causing components of the blood of the patient in the primary arterial vessel upstream the flow diverting apparatus being directed to the primary arterial vessel downstream the flow diverting apparatus is greater than a proportion of the inflammation-causing components of the blood of the patient in the primary arterial vessel upstream the flow diverting apparatus being directed to the secondary branch vessel.

24. The method of claim 18, wherein the secondary branch vessel is a first secondary branch vessel, wherein the implanted flow diverting apparatus is positioned and configured such that a proportion of the inflammation-causing components of the blood of the patient in the primary arterial vessel upstream the flow diverting apparatus being directed to a second secondary branch vessel is greater than a proportion of the inflammation-causing components of the blood of the patient in the primary arterial vessel upstream the flow diverting apparatus being directed to the first secondary branch vessel, and wherein the second secondary branch vessel branches off of the primary arterial vessel and leads to a portion of the body of the patient less affected with the chronic inflammation than the portion of the body to which the first secondary branch leads.

25. The method of claim 18, wherein the inflammation-causing components of the blood are cells having a diameter equal to or greater than 8 μm.

26. The method of claim 18, wherein the flow diverting apparatus is constructed of a mesh material having a pore diameter size within a range of 8 to 70 μm.

27. The method of claim 18, wherein the flow diverting apparatus is constructed of a mesh material having a pore length in a range of about 4 μm to about 36 μm and a pore width in a range of about 8 μm to about 64 μm.

28. The method of claim 18, wherein a portion of the flow diverting apparatus tapers from a first cross section to a second cross section at an angle within a range of 10 to 90 degrees, wherein the angle is measured from an axis extending through a length of the flow diverting apparatus.

29. A method of treating and/or preventing lymphedema or chronic inflammation in a patient comprising:
implanting a flow diverting apparatus in an infrarenal aorta of the patient such that the flow diverting apparatus is positioned upstream orifices of right and left common iliac arteries,
wherein the implanted flow diverting apparatus is positioned and configured such that a greater proportion of blood or a greater proportion of inflammation-causing components of the blood of the patient, is directed from the infrarenal aorta to the orifice of the right common iliac artery or the left common iliac artery as compared to the orifice of the other of the right common iliac artery or the left common iliac artery, and
wherein the other of the right common iliac artery or the left common iliac artery leads to a lower limb of the patient affected with lymphedema or chronic inflammation.

30. The method of claim 29, wherein the flow diverting apparatus is implanted subsequent to lymphovenous bypass surgery of the lower limb affected with the lymphedema.

31. The method of claim 29, wherein the implanted flow diverting apparatus is positioned in the infrarenal aorta such that a most tapered section of the flow diverting apparatus is upstream an aortic bifurcation of the patient by a distance within a range of about 0.5 cm to about 7.0 cm.

32. The method of claim 29, wherein the flow diverting apparatus includes a crescent-shaped tapered portion, and wherein the implanted flow diverting apparatus is positioned such that a midline of the crescent-shaped tapered portion is rotated 10 to 90 degrees with respect to an anteroposterior midline of the infrarenal aorta.
